(19) United States
(12) Patent Application Publication
(10) Pub. No.: US 2009/0169652 A1
(43) Pub. Date: Jul. 2, 2009

(54) Personal Care Compositions

(76) Inventor: Rosemarie Osborne, Oxford, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/274,865

(22) Filed: Nov. 20, 2008

Related U.S. Application Data
(60) Provisional application No. 60/991,005, filed on Nov. 29, 2007.

(51) Int. Cl. A61K 36/889; A61K 36/16; A61K 127/00; A61K 131/00; A61Q 19/08; A61Q 5/00; A61P 17/00; A61K 8/97; G06Q 90/00

(52) U.S. Cl. 424/727; 424/752; 705/500

(57) ABSTRACT

Personal care compositions comprising an active component, use of such compositions, and methods of marketing such compositions. The personal care compositions can be applied topically, ingested orally, injected, or used as part of a regimen.

### CROSS REFERENCE TO RELATED APPLICATION

This application claims the benefit of U.S. Provisional Application No. 60/991,005 filed on Nov. 29, 2008.

### TECHNICAL FIELD

The present invention relates to personal care compositions comprising one or more active components and optionally one or more optional components. Such compositions are useful for regulating the condition of mammalian keratinous tissue (e.g., skin, hair, and/or nails).

### BACKGROUND

Many personal care products currently available to consumers are directed primarily to improving the health and/or physical appearance of the skin, hair, or nails. Among these skin, hair, or nail care products, many are directed to delaying, minimizing or even eliminating skin, hair, or nail changes typically associated with the aging or the environmental damage to human skin, hair, or nails. Numerous compounds have been described in the art as being useful for regulating skin, hair, or nail condition.

Skin, hair, and nails are subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin, hair, or nails. Whether extrinsic or intrinsic, these factors result in visible signs of skin, hair, and nail aging and environmental damage (e.g., such as sunlight damage, smoke damage, and damage from pollutants such as nitrogen oxides, sulfur oxides, ozone, and metals such as lead). To many people, the loss of the attractiveness of skin, hair, or nails is a reminder of the disappearance of youth. As a result, the maintenance of a youthful appearance has become a booming business in youth-conscious societies. Numerous products and treatments are available in various forms to help maintain the appearance of younger hair, skin, and nails.

Extrinsic or intrinsic factors may result in the thinning and general degradation of the skin, hair, or nails. For example, as the skin, hair, and nails naturally age, there is a reduction in the cells and blood vessels that supply the skin, hair, or nails. There is also a flattening of the dermal-epidermal junction which results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990.

A large number of skin, hair, and nail care actives are known in the art and used to improve the health and/or cosmetic appearance of the skin, hair, or nails. However, a need still exists for personal care compositions that can provide the desired benefits and can be dermopharmaceutically and/or cosmetically preferred for particular applications.

### SUMMARY

The present invention provides personal care compositions comprising one or more active components that can help improve the health and/or cosmetic appearance of the skin, hair, or nails

The personal care compositions comprise one or more of such active components and/or derivatives of such active components, preferably in a safe and effective amount.

The present invention also relates to methods of using such compositions to regulate the condition of mammalian keratinous tissue (e.g., skin, hair, or nails). Said methods generally comprise the step of topically applying a composition of the present invention to the keratinous tissue (e.g., skin, hair, or nails) of a mammal in need of such treatment and/or to the keratinous tissue of a mammal desiring such treatment.

In another aspect, the method comprises the step of orally ingesting the active component, preferably a safe and effective amount of the active component, to regulate the condition of mammalian keratinous tissue (e.g., skin, hair, or nails). In one embodiment, the method comprises a dual treatment regimen comprising both oral ingestion of a composition and topical application of a composition, wherein at least one of the compositions comprises an active component according to the present invention.

In another aspect, the method comprises the step of injecting the active component, preferably injecting the active component into and/or under the skin. In a particular embodiment, the method comprises a treatment regimen comprising a combination of injection and/or oral administration and/or topical application.

These and other features, aspects, and advantages of the present invention will become evident to those skilled in the art from a reading of the present disclosure.

### DETAILED DESCRIPTION

While the specification concludes with the claims particularly pointing out and distinctly claiming the invention, it is believed that the present invention will be better understood from the following description.

As used herein, the term “active component” means one or more material that can regulate the condition of mammalian keratinous tissue, and is broad enough to include combinations thereof, as well as derivatives of active components.

All percentages and ratios used herein are by weight of the total composition and all measurements made are at 25° C., unless otherwise designated.

The term “keratinous tissue,” as used herein, refers to keratin-containing layers disposed as the outermost protective covering of mammals (e.g., humans, dogs, cats, etc.) which includes, but is not limited to, skin, mucosa, lips, hair, toenails, fingernails, cuticles, hooves, etc.

The terms “topical application”, “topically”, and “topical”, as used herein, mean to apply (e.g., spread, spray) the compositions of the present invention onto the surface of the keratinous tissue.

The terms “oral”, “orally”, and “oral administration”, as used herein, refer to orally ingesting a composition of the present invention.

The term “dermatologically acceptable,” as used herein, means that the compositions or components thereof so described are suitable for use in contact with mammalian keratinous tissue without undue toxicity, incompatibility, instability, allergic response, and the like.

The term “orally acceptable”, as used herein, means that the compositions or components thereof so described are suitable for oral ingestion by a mammal without undue toxicity, incompatibility, instability, allergic response, and the like.

As used herein, “effective amount” means an amount of a compound or composition sufficient to significantly induce a positive keratinous tissue benefit, including independently or in combination with other benefits disclosed herein. This means that the content and/or concentration of active component in the formulation is sufficient that when the formulation is applied with normal frequency and in a normal amount, the formulation can result in the treatment of one or more undesired keratinous tissue conditions (e.g., skin wrinkles). For instance, the amount can be an amount sufficient to inhibit or enhance some biochemical function occurring within the keratinous tissue. This amount of active component may vary depending upon the type of product, the type of keratinous tissue condition to be addressed, and the like.

The term “safe and effective amount” as used herein means an amount of a compound or composition sufficient to significantly induce a positive benefit, preferably a positive keratinous tissue appearance or feel benefit, including independently or in combinations with the benefits disclosed herein, but low enough to avoid serious side effects, i.e., to provide a reasonable benefit to risk ratio, within the scope of sound judgment of the skilled artisan.

The personal care compositions of the present invention can be useful for treating keratinous tissue (e.g., hair, skin, or nails) condition. As use herein, “treating” or “treatment” or “treat” includes regulating and/or immediately improving keratinous tissue cosmetic appearance and/or feel. As used herein, “regulating” or “regulation” means maintaining or improving the health and/or cosmetic appearance, and includes both prophylactically regulating and/or therapeutically regulating. Regulation of keratinous tissue condition, namely mammalian and in particular human skin, hair, or nail condition, is often required due to conditions which may be induced or caused by factors internal and/or external to the body. Examples include environmental damage, radiation exposure (including ultraviolet radiation), chronological aging, menopausal status (e.g., post-menopausal changes in skin, hair, or nails), stress, diseases, disorders, etc. For instance, “regulating skin, hair, or nail condition” includes prophylactically regulating and/or therapeutically regulating skin, hair, or nail condition, and may involve one or more of the following benefits: thickening of skin, hair, or nails (e.g., building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair.

As used herein, prophylactically regulating keratinous tissue condition includes delaying, minimizing and/or preventing visible and/or tactile discontinuities in keratinous tissue (e.g., texture irregularities in the skin, hair, or nails which may be detected visually or by feel), including signs of skin, hair, or nail aging. This is also encompassed within the term “treating.”

As used herein, therapeutically regulating keratinous tissue condition includes ameliorating, e.g., diminishing, minimizing and/or effacing, discontinuities in keratinous tissue (e.g., skin, hair, or nails). This is also encompassed within the term “treating.”

As used herein, “personal care composition” includes any product applied topically to keratinous tissue and/or ingested orally for the purpose of treating keratinous tissue (e.g., skin, hair, nails).

The compositions of the present invention can also be useful for immediately improving keratinous tissue (e.g., skin, hair, or nail) cosmetic appearance and/or feel. For example, topical compositions of the present invention can be useful for regulating the cosmetic appearance of skin, hair, or nail condition by providing an immediate visual improvement in skin, hair, or nail appearance following application of the composition to the skin, hair, or nails. Generally speaking, topical compositions of the present invention which further contain particulate materials (e.g., pigments) can be most useful for providing immediate visual improvement.

The term “sagging” as used herein means the laxity, slackness, or the like condition of skin that occurs as a result of loss of, damage to, alterations to, and/or abnormalities in dermal elastin, muscle and/or subcutaneous fat.

The terms “smoothing” and “softening” as used herein mean altering the surface of the keratinous tissue such that its tactile feel is improved.

“Signs of keratinous tissue aging” include, but are not limited to, all outward visibly and tactilely perceptible manifestations as well as any other macro or micro effects due to keratinous tissue aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin.

Compositions of the present invention are described in detail hereinafter.

### I. Personal Care Compositions

The personal care compositions of the present invention can comprise:

(1) an active component;

(2) an orally or dermatologically or injectibally acceptable carrier; and

(3) optionally, optional components.

The personal care compositions of the present invention can be in any suitable form. All forms of topical and oral personal care compositions comprising active components are contemplated and can include, for instance, creams, gels, lotions, emulsions, colloids, solutions, suspensions, ointments, milks, sprays, capsules, tablets, liquids, sticks, solids, powders, compacts, pencils, spray-on formulations, brush-on formulations, cloths, wipes, and the like.

Non-limiting examples of topical personal care compositions can include, without limitation, lipstick, mascara, rouge, foundation, blush, eyeliner, lipliner, lip gloss, facial or body powder, sunscreens and blocks, nail polish, mousse, sprays, styling gels, nail conditioner, bath and shower gels, shampoos, conditioners, cream rinses, hair dyes and coloring products, leave-on conditioners, sunscreens and sunblocks, lip balms, skin conditioners, cold creams, moisturizers, hair sprays, soaps, body scrubs, exfoliants, astringents, depilatories and permanent waving solutions, antidandruff formulations, antisweat and antiperspirant compositions, shaving, preshaving and after shaving products, moisturizers, deodorants, cold creams, cleansers, skin gels, and rinses. Furthermore, the composition can be applied topically through the use of a patch or other delivery device. Delivery devices can include, but are not limited to, those that can be heated or cooled, as well as those that utilize iontophoresis or ultrasound.

Non-limiting examples of oral personal care compositions can include, without limitation, tablets, pills, capsules, drinks, beverages, powders, vitamins, supplements, health bars, candies, chews, and drops.

Any desired suitable optional ingredients can be included in the personal care composition.

In a particular embodiment, the composition does not comprise substituted indole. In another embodiment, the composition does not comprise indole. In still another embodiment, the composition is substantially free of indole. In yet another embodiment, the composition is substantially free of substituted indole.

In another aspect, the present invention provides a personal care regimen comprising the use of at least one topical composition in combination with at least one oral composition. At least one of the compositions in this regimen comprises an active component according to the present invention. Preferably, the regimen includes at least one topical composition comprising such active component and at least one oral composition comprising such active component.

In another aspect, the method comprises the step of injecting the active component, preferably injecting the active component into and/or under the skin. In a particular embodiment, the method comprises a treatment regimen comprising a combination of injection and/or oral administration and/or topical application of the active component of the present invention.

In a further embodiment, the method comprises topically applying the composition everyday. In yet another embodiment, the method comprises topically applying the composition at night. In a particular embodiment, the method comprises topically applying the composition before going to bed, preferably at night or before retiring for the day. In still another embodiment, the method comprises: (a) cleansing a keratinous surface to form a cleansed keratinous surface; and (b) topically applying the composition to said cleansed keratinous surface.

### II. Active Components And Lipogenesis

The compositions of the present invention comprise an active component that can inhibit the synthesis of lipids in skin. Recent clinical studies by applicant has shown that inhibition of lipid synthesis in skin is directly related to anti-aging benefits. For instance, in one study, the oil control agent and preservative, sodium dehydroacetic acid (SDA), produced expected improvements in skin condition related to reduced oil, but also caused an unexpected improvement in the appearance of fine lines and wrinkles. Furthermore, it was shown that SDA up-regulated collagen expression in both Xgene and MatTek human skin equivalent culture models. Although not being limited by theory, it is believed by applicants that SDA may be an HMGCR inhibitor, producing skin anti-aging benefits through the inhibition of protein prenylation.

Based on the evidence from this study, it is hypothesized that inhibition of cholesterol/isoprenoid biosynthesis will provide an approach to improve the appearance of wrinkled photo-damaged skin. More specifically, inhibition of key steps in the cholesterol pathway can inhibit protein prenylation, which will lead to an increase in TGF-beta signaling, and an up-regulation of collagen gene expression in skin. Furthermore, it was predicted that SDA would inhibit cholesterol biosynthesis in skin.

As used herein, the term “active component” is broad enough to include one or more active components, one or more derivatives of active components, and combinations thereof. Preferably, the compositions comprise a safe and effective amount of such active component.

In one embodiment, the active component can be selected from the group consisting of: Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root Extract

Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof.

In one embodiment, the personal care composition can comprise the active component at a level of from about 0.000001% to about 10%, in another embodiment from about 0.0001% to about 5%, and in yet another embodiment from about 0.001% to about 1%, by weight of the entire composition.

The active components in accordance with the present invention, when provided in personal care compositions, are preferably provided in an amount which is safe and effective to treat at least one sign of an undesired keratinous tissue (e.g., skin, hair, or nail) condition. The phrase “to treat at least one undesired keratinous tissue (e.g., skin, hair, or nail) condition” as used herein means that the active component provides an objectively measurable increase in its effect on some aspect of the keratinous tissue (e.g., skin, hair, or nail) condition when used topically and/or orally and/or subcutaneously in an effective amount. This can be, for example, a greater reduction in the appearance of fine lines and wrinkles, increased potency, the ability to stimulate or inhibit at least one biochemical process within the skin, hair, or nails to a greater degree, increased strength of skin, increased firmness of skin, reduction of sebaceous gland size, reduction of follicular pore size, and the like. Generally, this is determined based on comparison to a control.

### III. Optional Components/Ingredients

The compositions of the present invention can comprise one or more suitable desired optional components. For example, the composition can optionally include other active or inactive ingredients.

For instance, the present invention may include additional skin care actives selected from the group consisting of sugar amines, vitamin B3, retinoids, peptides, dialkanoyl hydroxyproline, hexamidine, salicylic acid, phytosterol, sunscreen actives, water soluble vitamins, oil-soluble vitamins, their derivatives, their precursors, and combinations thereof.

For example, the compositions comprising the combination of active component and an additional keratinous tissue active, such as niacinamide, can be capable of providing additive and/or synergistic keratinous tissue (e.g., skin, hair, or nail) benefits.

Any other suitable optional component can also be included in the personal care composition of the present invention, such as those ingredients that are conventionally used in given product types. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, (e.g. hydroquinone, kojic acid, ascorbic acid, magnesiuim ascorbyl phosphate, ascorbyl glucoside, pyridoxine), skin-conditioning agents (e.g. humectants and occlusive agents), skin soothing and/or healing agents and derivatives (e.g. panthenol, and derivatives such as ethyl panthenol, aloe vera, pantothenic acid and its derivatives, allantoin, bisabolol, and dipotassium glycyrrhizinate), skin treating agents (e.g. vitamin D compounds, mono-,di-, and tri-terpenoids, beta-ionol, cedrol), thickeners, and vitamins and derivatives thereof.

Several preferred optional components are discussed in more detail below.

1. Sugar Amines (Amino Sugars)

The compositions of the present invention optionally include a safe and effective amount of a sugar amine, which are also known as amino sugars. The sugar amine compounds useful in the present invention are described in PCT Publication WO 02/076423 and U.S. Pat. No. 6,159,485.

Preferably, the composition contains from about 0.01% to about 15%, more preferably from about 0.1% to about 10%, and even more preferably from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

2. Vitamin B3

The compositions of the present invention may include a safe and effective amount of a vitamin B3 compound. Vitamin B3 compounds are particularly useful for regulating skin condition as described in U.S. Pat. No. 5,939,082. Preferably, the composition contains from about 0.01% to about 50%, more preferably from about 0.1% to about 20%, even more preferably from about 0.5% to about 10%, and still more preferably from about 1% to about 7%, even more preferably from about 2% to about 5%, by weight of the composition, of the vitamin B3 compound.

As used herein, “vitamin B3 compound” means a compound having the formula:

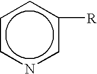

wherein R is —CONH2 (i.e., niacinamide), —COOH (i.e., nicotinic acid) or —CH2OH (i.e., nicotinyl alcohol); derivatives thereof, and salts of any of the foregoing.

Exemplary derivatives of the foregoing vitamin B3 compounds include nicotinic acid esters, including non-vasodilating esters of nicotinic acid (e.g., tocopheryl nicotinate, myristyl nicotinate).

Examples of suitable vitamin B3 compounds are well known in the art and are commercially available from a number of sources (e.g., the Sigma Chemical Company, ICN Biomedicals, Inc., and Aldrich Chemical Company). A preferred vitamin B3 compound useful in the present invention is niacinamide.

3. Retinoid

The compositions of this invention may contain a safe and effective amount of a retinoid, such that the resultant composition is safe and effective for regulating keratinous tissue condition, preferably for regulating visible and/or tactile discontinuities in skin, more preferably for regulating signs of skin aging. The compositions preferably contain from about 0.001% to about 10%, more preferably from about 0.005% to about 2%, even more preferably from about 0.01% to about 1%, still more preferably from about 0.01% to about 0.5%, by weight of the composition, of the retinoid. The optimum concentration used in a composition will depend on the specific retinoid selected since their potency does vary considerably.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C2-C22 alkyl esters of retinol, including retinyl palmitate, retinyl acetate, retinyl propionate), retinal, and/or retinoic acid (including all-trans retinoic acid and/or 13-cis-retinoic acid), or mixtures thereof. More preferably the retinoid is a retinoid other than retinoic acid. Preferred retinoids are retinol, retinyl palmitate, retinyl acetate, retinyl propionate, retinal and combinations thereof. More preferred is retinyl propionate, used even more preferably from about 0.1% to about 0.3%.

4. Peptide

The compositions of the present invention may contain a safe and effective amount of a peptide, including but not limited to, di-, tri-, tetra-, penta-, and hexa-peptides and derivatives thereof. The compositions contain preferably from about 1×10-6% to about 20%, more preferably from about 1×10-6% to about 10%, even more preferably from about 1×10-5% to about 5%, by weight of the composition.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. More preferred peptides are the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as Iamin). A preferred dipeptide derivative is palmitoyl-lys-thr. A preferred commercially available tripeptide derivative-containing composition is Biopeptide CL®, which contains 100 ppm of palmitoyl-gly-his-lys and is commercially available from Sederma. A preferred commercially available pentapeptide derivative-containing composition is Matrixyl®, which contains 100 ppm of palmitoyl-lys-thr-thr-lys-ser and is commercially available from Sederma.

5. Phytosterol

The topical compositions of the present invention comprise a safe and effective amount of one or more phytosterols selected from the group consisting of β-sitosterol, campesterol, brassicasterol, A5-avennasterol, lupenol, α-spinasterol, stigmasterol, their derivatives, analogs, and combinations thereof. More preferably, the phytosterol is selected from the group consisting of β-sitosterol, campesterol, brassicasterol, stigmasterol, their derivatives, and combinations thereof. More preferably, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

In the compositions of the present invention, the phytosterol preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2% by weight of the composition.

6. Hexamidine

The hexamidine compounds useful in the present invention correspond to those of the following chemical structure:

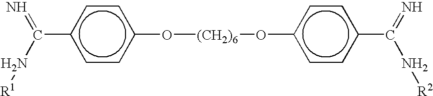

wherein R1 and R2 comprise organic acids (e.g., sulfonic acids, etc.).

In the composition of the present invention, the hexamidine preferably comprises from about 0.0001 to about 25%, more preferably from about 0.001 to about 10%, more preferably from about 0.01 to about 5%, and even more preferably from about 0.02 to about 2.5% by weight of the composition.

The topical compositions of the present invention optionally include a safe and effective amount of one or more of hexamidine compounds, its salts, and its derivatives. As used herein, hexamidine derivatives include any isomers and tautomers of hexamidine compounds including but not limited to organic acids and mineral acids, for example sulfonic acid, carboxylic acid etc. Preferably, the hexamidine compounds include hexamidine diisethionate, commercially available as Eleastab® HP100 from Laboratoires Serobiologiques.

7. Dehydroacetic Acid (DHA)

The composition of this invention can include dehydroacetic acid, having the structure:

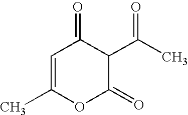

or pharmaceutically acceptable salts, derivatives or tautomers thereof. As used herein, “pharmaceutically acceptable” means that the salts of dehydroacetic acid are suitable for use in contact with the tissues of mammals to which they will be exposed without undue toxicity, incompatibility, instability, irritation, allergic response, and the like. The technical name for dehydroacetic acid is 3-Acetyl-6-methyl-2H-pyran-2,4(3H)-dione and can be commercially purchased from Lonza.

Pharmaceutically acceptable salts include alkali metal salts, such as sodium and potassium; alkaline earth metal salts, such as calcium and magnesium; non-toxic heavy metal salts; ammonium salts; and trialkylammonium salts, such astrimethylammonium and triethylammonium. Sodium, potassium, and ammonium salts of dehydroacetic acid are preferred. Highly preferred is sodium dehydroacetate which can be purchased from Tri-K, as Tristat SDHA. Derivatives of dehydroacetic acid include, but are not limited to, any compounds wherein the CH3 groups are individually or in combination replaced by amides, esters, amino groups, alkyls, and alcohol esters. Tautomers of dehydroacetic acid are the isomers of dehydroacetic acid which can change into one another with great ease so that they ordinarily exist in equilibrium. Thus, tautomers of dehydroacetic acid can be described as having the chemical formula C8H8O4 and generally having the structure above.

In one embodiment, the compositions of the present invention can comprise from about 0.001% to about 25% by weight of the composition, preferably from about 0.01% to about 10%, more preferably from about 0.05% to about 5%, and even more preferably from about 0.1% to about 1%, of dehydroacetic acid or pharmaceutically acceptable salts, derivatives or tautomers thereof.

8. Dialkanoyl Hydroxyproline Compounds

The dialkanoyl hydroxyproline compounds of the present invention correspond to those of the following chemical structure:

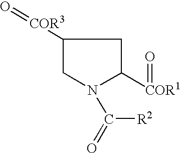

wherein R1 comprises H, X, C1-C20 straight or branched alkyl,
X comprises metals (Na, K, Li, Mg, Ca) or amines (DEA, TEA);
R2 comprises C1-C20 straight or branched alkyl;
R3 comprises C1-C20 straight or branched alkyl.

The topical compositions of the present invention may comprise a safe and effective amount of one or more dialkanoyl hydroxyproline compounds and their salts and derivatives. In the composition of the present invention, the dialkanoyl hydroxyproline compounds preferably comprise from about 0.01 to 10%, more preferably from about 0.1-5%, even more preferably from about 0.1 to 2% by weight of the composition

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably the dipalmitoyl hydroxyproline is the triethanolamine salt of dipalmitoyl hydroxyproline.

9. Salicylic Acid Compound

The topical compositions of the present invention may comprise a safe and effective amount of a salicylic acid compound, its esters, its salts, or combinations thereof. In the compositions of the present invention, the salicylic acid compound preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2%, by weight of the composition, of salicylic acid.

10. N-acyl Amino Acid Compound

The topical compositions of the present invention comprise a safe and effective amount of one or more N-acyl amino acid compounds. The amino acid can be one of any of the amino acids known in the art. The N-acyl amino acid compounds of the present invention correspond to the formula:

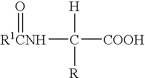

wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W.H. Freeman and Company. R1 can be C1 to C30, saturated or unsaturated, straight or branched, substituted or unsubstituted alkyls; substituted or unsubstituted aromatic groups; or mixtures thereof. Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof. N-acyl Phenylalanine corresponds to the following formula:

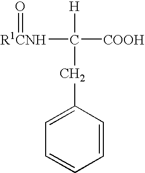

wherein R1 can be C1 to C30, saturated or unsaturated, straight or branched, substituted or unsubstituted alkyls; substituted or unsubstituted aromatic groups; or mixtures thereof. N-acyl Tyrosine corresponds to the following formula:

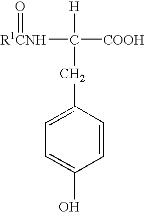

wherein R1 can be C1 to C30, saturated or unsaturated, straight or branched, substituted or unsubstituted alkyls; substituted or unsubstituted aromatic groups; or mixtures thereof. Particularly useful as a topical skin tone evening (lightening or pigmentation reduction) cosmetic agent is N-undecylenoyl-L-phenylalanine. This agent belongs to the broad class of N-acyl Phenylalanine derivatives, with its acyl group being a C11 mono-unsaturated fatty acid moiety and the amino acid being the L-isomer of phenylalanine. N-undecylenoyl-L-phenylalanine corresponds to the following formula:

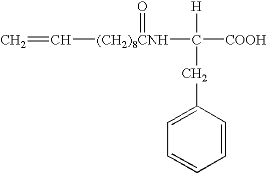

As used herein, N-undecylenoyl-L-phenylalanine is commercially available under the tradename Sepiwhite® from SEPPIC.

In the composition of the present invention, the N-acyl amino acid preferably comprises from about 0.0001-25%, more preferably from about 0.001-10%, more preferably from about 0.01-5%, and even more preferably from about 0.02-2.5% by weight of the composition.

11. Hesperedin

The compositions of the present invention may include a safe and effective amount of hesperedin. Preferably, the composition contains from about 0.01% to about 10%, more preferably from about 0.1% to about 5%, even more preferably from about 0.5% to about 3%, by weight of the composition, of the hesperedin compound.

Hesperedin is a flavonoid. Oxygen radicals are produced in the skin in response to many stimuli, such as exposure to UV and irritants. Such radicals are also produced as by-products of normal cell or tissue metabolism. Oxygen radicals can stimulate pigment cells (melanocytes) to increase production of melanin. Hesperidin has anti-oxidant properties and thus can scavenge oxygen radicals before they stimulate the melanocytes.

The protein tyrosinase is an enzyme involved in the conversion of the amino acid tyrosine to DOPA (dihydroxyphenylalanine) which then is further converted into other intermediates and polymerized into the skin pigment melanin. Partial or complete inhibition of tyrosinase slows or stops, respectively, the formation of melanin, leading to lighter skin color (e.g., reduction in darkness of hyperpigmented spots). Hesperidin also inhibits tyrosinase.

12. Mustard Seed Extract

The compositions of the present invention may include a safe and effective amount of mustard seed extract. Preferably, the composition contains from about 0.1% to about 20%, more preferably from about 0.5% to about 10%, even more preferably from about 1% to about 5%, by weight of the composition, of the mustard seed extract compound. A preferred mustard seed extract is Sinablanca®. Sinablanca® is hydrolyzed Brassica Alba seed extract and is believed to inhibit tyrosinase

13. Glycyrrhizic acid

The compositions of the present invention may include a safe and effective amount of glycyrrhizic acid. Preferably, the composition contains from about 0.01% to about 10%, more preferably from about 0.05% to about 5%, even more preferably from about 0.1% to about 3%, by weight of the composition, of the glycyrrhizic acid compound. Glycyrrhizic acid is a component of licorice extract.

Glycyrrhizic acid is an anti-inflammatory agent. Inflammatory mediators or cytokines can stimulate pigment cells (melanocytes) to produce melanin. Thus inflammatory conditions such as UV-damage, acne, in-grown hairs, insect bites, scratches, etc. will stimulate what is called post-inflammatory hyperpigmentation. While UV is a primary inducer of pigmentation in all skin types, pigment from the other inflammatory stimuli (acne, etc.) will in particular contribute to skin pigmentation in darker skin individuals (e.g., Hispanic, Asian). Inhibiting inflammation with anti-inflammatory agents will reduce pigmentation.

Glycyrrhizic acid is also believed to be a scavenger of nitric oxide. Nitric oxide (NO) is a stimulator of pigmentation. Use of nitric oxide scavengers (materials that react with nitric oxide to prevent it from stimulating pigment cells) will reduce pigmentation.

Glycyrrhizic acid is also known as glycyrrhizin, glycyrrhizinic acid, or glycyrrhetinic acid glycoside.

14. Glycyrrhetinic acid

The compositions of the present invention may include a safe and effective amount of glycyrrhetinic acid. Preferably, the composition contains from about 0.01% to about 10%, more preferably from about 0.05% to about 5%, even more preferably from about 0.1% to about 3%, by weight of the composition, of the glycyrrhetinic acid compound. Glycyrrhetinic acid is a component of licorice extract.

Glycyrrhetinic acid is also an anti-inflammatory agent, discussed above in the glycyrrhizic acid section. Structurally, glycyrrhetinic acid is different from glycyrrhizic acid in that glycyrrhetinic acid does not have an attached sugar residue (glycoside). Glycyrrhetinic acid is also known as enoxolone, glycyrrhetic acid, or uralenic acid.

15. Butylated Hydroxyanisole (BHA)

The compositions of the present invention may include a safe and effective amount of BHA. BHA is a mild anti-oxidant, and is often included in cosmetic preparations as a preservative.

BHA can be purchased from various suppliers, including Eastman Chemical (Kingsport, Tenn.), Alfa Chemical (Kings Point, N.Y.), and Shell Chemical Company (Houston, Tex.).

BHA may be present in an amount of from about 0.01% to about 10%, more preferably from about 0.05% to about 5%, more preferably from about 0.1% to about 3%, by weight of the composition.

16. Menthyl Anthranilate

The compositions of the present invention may comprise a safe and effective amount of menthyl anthranilate, its salt, and derivatives thereof. Menthyl anthranilate may be present in an amount of from about 0.01% to about 20% by weight of the composition, more preferably from about 0.1% to about 15% by weight of the composition, even more preferably from about 1% to about 10% by weight of the composition.

Menthyl anthranilate acts as a trypsin-type protease inhibitor. Such a protease is involved in the transfer of melanosomes (the small pigment-containing granules) from melanocytes (epidermal cells that produce pigment) to keratinocytes (the primary cell in the epidermis). When transfer is inhibited, the melanocytes stop producing more pigment. There are two forms of menthyl anthranilate: the +isomer and the −isomer. Both isomers and their combination are active as trypsin-type protease inhibitor. Thus, either isomer, or their combination, is useful in the compositions of this invention. Menthyl anthranilate is commercially available from Phoenix Aromas & Essential Oils, Inc. (Norwood, N.J.) and Alzo International (Sayreville, N.J.).

17. Cetyl Pyridinium Chloride (CPC)

The compositions of the present invention may comprise a safe and effective amount of cetyl pyridinium chloride (CPC). Alternate forms of cetyl pyridinium chloride include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl) ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds are bis-4-(R-amino)-1-pyridinium alkanes as disclosed in U.S. Pat. No. 4,206,215.

Cetyl pyridinium chloride may be present in an amount of from about 0.005% to about 10% by weight of the composition, more preferably from about 0.01% to about 5%, more preferably from about 0.05% to about 2%. Cetyl pyridinium chloride is an inhibitor of tyrosinase, a mechanism discussed above.

18. Ergothioneine

The compositions of the present invention may comprise a safe and effective amount of ergothioneine. Ergothioneine may be present in an amount of from about 0.01% to about 20% by weight of the composition, more preferably from about 0.1% to about 15% by weight of the composition, even more preferably from about 1% to about 10% by weight of the composition. A preferred ergothioneine is Thiotaine® which is a commercial solution of the chemical ergothioneine, commercially available from Barnet Products. Ergothioneine exhibits anti-oxidant properties, a mechanism described above.

19. Vanillin

The compositions of the present invention may comprise a safe and effective amount of vanillin or its derivatives. Vanillin may be present in an amount of from about 0.01% to about 20% by weight of the composition, more preferably from about 0.1% to about 15% by weight of the composition, even more preferably from about 0.5% to about 10% by weight of the composition.

A preferred vanillin derivative is vanillin acetate, which is believed to be a subtilisin-type protease inhibitor. The peptide hormone melanocyte stimulating hormone (MSH) induces pigment cells to make melanin. MSH is produced as a larger inactive precursor peptide (pro-hormone) which must be cleaved by a protease to the active MSH. That protease is a subtilisin-type protease.

Other related compounds useful in the present invention include vanillic acid, vanillin, o-vanillin, ethyl vanillin, isovanillin, vanillin methyl ether, vanillin ethyl ether, o-ethyl vanillin, vanillin oxime, vanillin benzyl ether, homovanillin, vanillin isobutyrate, divanillin, and isovanillin oxime.

20. Diethylhexyl Syrinylidene Malonate

The compositions of the present invention may comprise a safe and effective amount of diethylhexyl syrinylidene malonate. Diethylhexyl syrinylidene malonate may be present in an amount of from about 0.01% to about 20% by weight of the composition, more preferably from about 0.1% to about 15% by weight of the composition, even more preferably from about 0.5% to about 10% by weight of the composition.

A preferred diethylhexyl syrinylidene malonate is Oxynex® which exhibits anti-oxidant properties. It is available from Rona/Merck.

21. Melanostatine

The compositions of the present invention may comprise a safe and effective amount of melanostatine. Melanostatine may be present in an amount of from about 0.01% to about 20% by weight of the composition, more preferably from about 0.1% to about 15% by weight of the composition, even more preferably from about 0.5% to about 10% by weight of the composition. Since melanostatine is a commercial solution of peptide (approximately 50 ppm peptide in this commercial solution), the actual level of peptide in a product containing 5% melanostatine actually contains approximately 2.5 ppm peptide).

A preferred melanostatine is available from Vincience (France). Melanostatine is a hexapeptide, and it operates mechanistically by inhibiting binding of alpha-MSH (melanin stimulating hormone) to its cell receptor, thus inhibiting initiation of pigmentation.

22. Sterol Esters

The compositions of the present invention may comprise a safe and effective amount of sterol esters. The sterol esters may be present in an amount of from about 0.01% to about 20% by weight of the composition, more preferably from about 0.1% to about 15% by weight of the composition, even more preferably from about 0.5% to about 10% by weight of the composition.

When sterol esters are used in the present invention, formulation of the composition should be performed so that hydrolysis of the esters does not occur. Therefore, the ideal pH range of the composition comprising sterol esters is from about 3 to about 8, preferably from about 4 to about 7.

Sterol esters useful in the present invention may be comprised of sterols or mixtures of sterols (in particular sitosterol, campesterol, stigmasterol, brassicasterol, and additional sterols) which are esterified with a fatty acid or mixtures of fatty acids (which can be straight chain or branched chain, saturated or unsaturated) with from 8 to 30 carbon atoms (preferably 16-22 carbon atoms). Sterol esters are available from P&G Chemicals.

23. Sunscreen Actives

The compositions of the subject invention may optionally contain a sunscreen active. As used herein, “sunscreen active” includes both sunscreen agents and physical sunblocks. Suitable sunscreen actives may be organic or inorganic.

A wide variety of conventional sunscreen actives are suitable for use herein. Sagarin, et al., at Chapter VIII, pages 189 et seq., of Cosmetics Science and Technology (1972), discloses numerous suitable actives. Particularly suitable sunscreen agents are 2-ethylhexyl-p-methoxycinnamate (commercially available as PARSOL MCX), 4,4′-t-butyl methoxydibenzoylmethane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds.

Preferred organic sunscreen actives useful in the compositions of the present invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof. Especially preferred sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

The sunscreen active preferably comprises from about 1% to about 20%, more preferably from about 2% to about 10%, by weight of the composition. Exact amounts will vary depending upon the sunscreen chosen and the desired Sun Protection Factor (SPF).

24. Water-Soluble Vitamins

The compositions of the present invention may contain a safe and effective amount of one or more water-soluble vitamins. Examples of water-soluble vitamins include, but are not limited to, water-soluble versions of vitamin B (such as vitamin B5 and vitamin B6), vitamin B derivatives, vitamin C (such as ascorbyl glucoside), vitamin C derivatives (such as magnesium ascorbyl phosphate, sodium ascorbyl phosphate, and ascorbyl palmitate), vitamin K, vitamin K derivatives, pro-vitamins thereof, such as panthenol and mixtures thereof. When vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the vitamin compound.

25. Oil-Soluble Vitamins

The compositions of the present invention may contain a safe and effective amount of one or more oil-soluble vitamins. Examples of oil-soluble vitamins include, but are not limited to, oil-soluble versions of vitamin D, vitamin D derivatives, vitamin E (such as vitamin E acetate), vitamin E derivatives, pro-vitamins thereof, and mixtures thereof. When oil-soluble vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the oil-soluble vitamin compound.

26. Other actives

The compositions of the present invention may contain a safe and effective amount of one or more of the following other actives or ingredients: fatty acids (especially poly-unsaturated fatty acids), glucosamine, zinc pyrithione (ZPT), anti-fungal agents, thiol compounds (e.g., N-acetyl cysteine, glutathione, thioglycolate), other vitamins (vitamin B12), beta-carotene, ubiquinone, idebenone, amino acids, and the like.

### IV. Carrier

The compositions of the present invention can comprise an orally, dermatologically, or injection/subcutaneously acceptable carrier, depending upon the desired product form.

a. Dermatologically Acceptable Carrier

The topical compositions of the present invention can also comprise a dermatologically acceptable carrier for the composition. In one embodiment, the carrier is present at a level of from about 50% to about 99.99%, preferably from about 60% to about 99.9%, more preferably from about 70% to about 98%, and even more preferably from about 80% to about 95%, by weight of the composition.

The carrier can be in a wide variety of forms. Non-limiting examples include simple solutions (water or oil based), emulsions, and solid forms (gels, sticks). For example, emulsion carriers can include, but are not limited to, oil-in-water, water-in-oil, water-in-silicone, water-in-oil-in-water, and oil-in-water-in-silicone emulsions.

Depending upon the desired product form, preferred carriers can comprise an emulsion such as oil-in-water emulsions (e.g., silicone in water) and water-in-oil emulsions, (e.g., water-in-silicone emulsions). As will be understood by the skilled artisan, a given component will distribute primarily into either the water or oil phase, depending on the water solubility/dispensability of the component in the composition. In one embodiment, oil-in-water emulsions are especially preferred.

Emulsions according to the present invention can contain an aqueous phase and a lipid or oil. Lipids and oils may be derived from animals, plants, or petroleum and may be natural or synthetic (i.e., man-made). Preferred emulsions can also contain a humectant, such as glycerin. Emulsions can further comprise from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The compositions of the present invention can be in the form of pourable liquids (under ambient conditions). The compositions can therefore comprise an aqueous carrier, which is typically present at a level of from about 20% to about 95%, preferably from about 60% to about 85%. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

b. Orally Acceptable Carrier

The compositions of the present invention can also comprise an orally acceptable carrier if they are to be ingested. Any suitable orally ingestible carrier or carrier form, as known in the art or otherwise, can be used. Non-limiting examples of oral personal care compositions can include, but are not limited to, tablets, pills, capsules, drinks, beverages, powders, vitamins, supplements, health bars, candies, chews, and drops.

c. Injectible Liquid

The compositions of the present invention can also comprise a liquid that is acceptable for injection in and/or under the skin if the composition is to be injected. Any suitable acceptable liquid as known in the art or otherwise can be used.

### V. Composition Preparation

The compositions useful for the methods of the present invention are generally prepared by conventional methods such as are known in the art of making topical and oral compositions and compositions for injection. Such methods typically can involve mixing of the ingredients in one or more steps to a relatively uniform state, with or without heating, cooling, application of vacuum, and the like.

### VI. Methods for Treating Keratinous Tissue Condition

The compositions of the present invention can be useful for treating a number of mammalian keratinous tissue conditions. Such treatment of keratinous tissue conditions can include prophylactic and therapeutic regulation. More specifically, such treatment methods can be directed to, but are not limited to, preventing, retarding, and/or treating uneven skin tone, reducing the size of pores in mammalian skin, regulating oily/shiny appearance of mammalian skin, thickening keratinous tissue (i.e., building the epidermis and/or dermis and/or subcutaneous layers of the skin and where applicable the keratinous layers of the nail and hair shaft), preventing, retarding, and/or treating uneven skin tone by acting as a lightening or pigmentation reduction cosmetic agent, preventing, retarding, and/or treating atrophy of mammalian skin, softening and/or smoothing lips, hair and nails of a mammal, preventing, retarding, and/or treating itch of mammalian skin, preventing, retarding, and/or treating the appearance of dark under-eye circles and/or puffy eyes, preventing, retarding, and/or treating sallowness of mammalian skin, preventing, retarding, and/or treating sagging (i.e., glycation) of mammalian skin, preventing and/or retarding tanning of mammalian skin, desquamating, exfoliating, and/or increasing turnover in mammalian skin, preventing, retarding, and/or treating hyperpigmentation such as post-inflammatory hyperpigmentation, preventing, retarding, and/or treating the appearance of spider vessels and/or red blotchiness on mammalian skin, preventing, retarding, and/or treating fine lines and wrinkles of mammalian skin, preventing, retarding, and/or treating skin dryness (i.e., roughness, scaling, flaking) and preventing, retarding, and/or treating the appearance of cellulite in mammalian skin. In a preferred embodiment, the composition is used to treat the signs of aging; in one aspect, the composition is used to regulate the signs of aging; in another aspect, the composition is used to reduce or decrease the signs of aging; in yet another aspect the composition is used to prevent the signs of aging in keratinous tissue (e.g., skin, hair, or nails).

For instance, the present invention can be useful for therapeutically regulating visible and/or tactile discontinuities in mammalian keratinous tissue, including discontinuities in skin texture and color. For example, the apparent diameter of pores can be decreased, the apparent height of tissue immediately proximate to pore openings can approach that of the interadnexal skin, the skin tone/color can become more uniform, and/or the length, depth, and/or other dimension of lines and/or wrinkles can be decreased. Furthermore, compositions of the present invention can also be useful for cleansing (e.g, hair, body, facial), improving keratinous tissue feel (wet & dry) such as for hair (e.g., improving appearance/look, detangling, shine, gloss, decrease coefficient of friction, increase smoothness, color retention, decrease split ends, prevent hair breakage, prevent environmental damage such as sunlight damage, smoke damage, and damage from pollutants such as nitrogen oxides, sulfur oxides, ozone, and metals such as lead), odor control, oil control, conditioning, hair volume control, hair growth, and hair growth inhibition.

Regulating keratinous tissue conditions can involve topically applying to the keratinous tissue a safe and effective amount of a composition of the present invention. The amount of the composition that is applied, the frequency of application, and the period of use will vary widely depending upon the level of components of a given composition and the level of regulation desired, e.g., in view of the level of keratinous tissue damage present or expected to occur.

Furthermore, regulating keratinous tissue conditions can involve orally ingesting a safe and effective amount of a composition of the present invention. The amount of the composition that is ingested, the frequency of ingestion, and the period of use will vary widely depending upon the level of components of a given composition and the level of regulation desired, e.g., in view of the level of keratinous tissue damage present or expected to occur.

In one embodiment, the composition is chronically applied to the skin, e.g. topically. By “chronic application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however, application rates can vary, and can include from about once per week up to about three times per day or more.

A wide range of quantities of the compositions of the present invention can be employed to provide a keratinous tissue appearance and/or feel benefit when applied topically. For example, quantities of the present compositions, which are typically applied per application are, in mg composition/cm² keratinous tissue, from about 0.1 mg/cm² to about 20 mg/cm². A particularly useful application amount is about 0.5 mg/cm² to about 10 mg/cm².

Treating keratinous tissue condition can be practiced, for example, by applying a composition in the form of a skin lotion, clear lotion, milky lotion, cream, gel, foam, ointment, paste, emulsion, spray, conditioner, tonic, cosmetic, lipstick, foundation, nail polish, after-shave, or the like which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, (e.g., face, lips, under-eye area, eyelids, scalp, neck, torso, arms, hands, legs, feet, fingernails, toenails, scalp hair, eyelashes, eyebrows, etc.) The application of the present compositions may be done using the palms of the hands and/or fingers or a device or implement (e.g., a cotton ball, swab, pad, applicator pen, spray applicator, etc.)

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows feet area, frown lines, under eye area, upper lip, and the like). The patch can be occlusive, semi-occlusive or non-occlusive, and can be adhesive or non-adhesive. The composition can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can also contain a source of electrical energy (e.g., a battery) to, for example, increase delivery of the composition and active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

In another embodiment, a personal care regimen is used to regulate the condition of keratinous tissue. By “regimen” is meant the use of an oral composition in conjunction with a topical composition. In a particular embodiment, the oral composition and the topical composition are packaged together as a kit. In another embodiment, the oral composition and the topical composition are not packaged together as a kit, but potential users of the regimen are informed (e.g. through advertisements, product labeling) that the oral and the topical compositions may be used in conjunction with one another to regulate the condition of kerationous tissue. At least one of the compositions, either oral or topical, comprise an active component of the present invention. Preferably, both the oral and the topical compositions comprise an active component of the present invention.

### VII. Method of Doing Business Method of Marketing

In another aspect, the present invention provides a method of doing business wherein said method of doing business comprises a method of marketing a personal care composition. In one embodiment, the method of doing business comprises a method of marketing a personal care composition comprising an active component, wherein said method of marketing comprises:

(a) making available for sale to a potential customer a personal care composition comprising an active component;

(b) communicating to said potential customer that said composition can be helpful in regulating a keratinous tissue condition.

In another embodiment, the method of marketing comprises:

(a) making available for sale to a potential customer a first personal care composition;

(b) communicating to said potential customer a comparison of said first personal care composition to a second personal care composition, where said second personal care composition comprises an active component.

In still another embodiment, the method of marketing comprises:

(a) providing to a potential customer an article of commerce, wherein said article of commerce comprises:
(1) a container;
(2) a personal care composition contained in said container;
(3) graphics comprising at least two colors on said container;

(b) communicating to said potential customer that said article of commerce is similar to a second article of commerce, wherein said second article of commerce comprises:
(1) a second container;
(2) a second personal care composition contained in said second container, wherein said second personal care composition comprises an active component, wherein said active component is selected from the group consisting of In one embodiment, the active component can be selected from the group consisting of: Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root Extract
   Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof;
(3) a second set of graphics disposed upon said second container, wherein said a second set of graphics comprises at least one of the same colors as those colors included in said first set of graphics.

In yet another embodiment, the method comprises:

(a) providing to a potential customer an article of commerce, wherein said article of commerce comprises:
(1) a container;
(2) a personal care composition contained in said container;
(3) graphics comprising at least two colors on said container;

(b) locating said article of commerce within close proximity to a second article of commerce, wherein said second article of commerce comprises:
(1) a second container;
   (2) a second personal care composition contained in said container, wherein said second personal care composition comprises an active component, wherein said active component is selected from the group consisting of:
   Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root Extract
   Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof; and
(3) a second set of graphics disposed upon said second container, wherein said second set of graphics comprises at least two of the same colors as those colors included in said first set of graphics.

In a further embodiment, the method comprises:

(a) providing to a potential customer a personal care composition comprising an active component;

(b) communicating to said potential customer that said composition can be helpful in regulating keratinous tissue condition, wherein said keratinous tissue condition is selected from the group consisting of hair growth promotion, hair thickness, hair density, the natural process of skin again and damage, nail growth, and nail density.

### Lipogenesis Assay

In the Lipogenesis Assay, pre-adipocyte cells are cultured in PM-1 medium at a density of 12,500 cells per well in 96 well clear bottom white plates for 24-48 hours. The culture medium is then changed to differentiation medium (DM-2) and the cells grown for 6 days. The cells are then treated with, usually, an 100 uM concentration of test compound and grown for an additional 6 days. The cells are then stained with Adipored reagent to determine the extent of lipogenesis inhibition.

### EXAMPLES

The following are non-limiting examples of compositions of the present invention. The examples are given solely for the purpose of illustration and are not to be construed as limitations of the present invention, as many variations thereof are possible without departing from the spirit and scope of the invention, which would be recognized by one of ordinary skill in the art.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples 1-5

### Moisturizing Oil-In-Water Lotions/Creams

| | | | | |
|---|---|---|---|---|
| 1 | 2 | 3 | 4 | 5 |

Water Phase:

| | | | | | |
|---|---|---|---|---|---|
| Water | QS | QS | QS | QS | QS |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | — | — | 0.5 | — | — |
| Benzyl alcohol | 0.25 | 0.25 | 0.25 | 0.25 | 0.25 |
| GLW75CAP-MP (75% aq. | — | 0.5 | 0.5 | — | — |
| TiO2 dispersion)¹ | | | | | |
| Hexamidine diisethionate | — | 0.1 | — | 0.1 | — |
| Palmitoyl-peptide² | 0.00055 | 0.00055 | 0.0001 | 0.0003 | 0.00055 |
| N-acetyl glucosamine | 2 | 1 | 2 | 2 | 1 |
| 1,2-Hexanediol | 1 | 3 | — | — | — |
| 1,2-Pentanediol | — | 0.1 | — | 1 | — |
| Hexa-peptide gly-pro-gln-gly- | 0.1 | 0.5 | 0.5 | — | 1 |
| pro-gln (hexa-peptide-9) | | | | | |

Oil Phase:

| | | | | | |
|---|---|---|---|---|---|
| Tetrahydrocucurmin | — | 0.5 | — | 1 | — |
| Salicylic Acid | — | — | 1.5 | — | — |
| Isohexadecane | 3 | 3 | 3 | 4 | 3 |
| PPG15 Stearyl Ether | — | 5 | 8 | 10 | 2 |
| Isopropyl Isostearate | 1 | 0.5 | 1.3 | 1.5 | 1.3 |
| Sucrose polyester | 0.7 | — | 0.7 | 1 | 0.7 |
| Dipalmitoylhydroxyproline | — | — | — | 1.0 | — |
| Undecylenoyl Phenylalanine | — | 0.5 | — | — | — |
| Phytosterol | — | — | 0.5 | — | 0.1 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |
| Ginkgo Biloba Leaf Extract | — | — | 0.1 | 1 | 2 |
| Date palm kernal extract | 4 | 4 | 2 | — | — |

Thickener:

| | | | | | |
|---|---|---|---|---|---|
| Polyacrylamide/C13-14 | 1.5 | — | 2 | 2.5 | 2 |
| isoparaffin/laureth-7 | | | | | |
| Polyquaternium-37 | — | 2 | — | — | — |

Additional Ingredients:

| | | | | | |
|---|---|---|---|---|---|
| Dimethicone/dimethiconol | — | 1 | 2 | 0.5 | 2 |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

¹Available from Kobo products
²Available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (e.g., with a Tekmar T-25). Then, add the thickener to the emulsion and cool the emulsion to 45° C. while stirring. At 45° C., add the remaining ingredients. Cool the product and stir to 30° C. and pour into suitable containers.

### Examples 6-11

### Moisturizing Silicone-In-Water Serums/Lotions

| | | | | | |
|---|---|---|---|---|---|
| 6 | 7 | 8 | 9 | 10 | 11 |

Water Phase:

| | | | | | | |
|---|---|---|---|---|---|---|
| Water | Qs | Qs | Qs | Qs | Qs | Qs |
| Glycerin | 3 | 5 | 7 | 10 | 15 | 10 |
| Disodium EDTA | 0.1 | 0.1 | 0.05 | 0.1 | 0.1 | 0.1 |
| Niacinamide | 2 | 0.5 | — | 3 | 5 | 3 |
| Sodium | — | — | 0.1 | — | — | — |
| Dehydroacetate | | | | | | |
| Sodium PEG-7 Olive | 0.1 | — | 0.1 | — | 0.1 | — |
| Oil Carboxylate | | | | | | |
| D-panthenol | 0.5 | 0.1 | | 0.5 | 1.5 | 0.5 |
| GLW75CAP-MP | — | 0.4 | — | — | — | 0.4 |
| (75% aq. TiO2 | | | | | | |
| dispersion)¹ | | | | | | |
| Ascorbyl Glucoside | — | — | — | — | — | 1 |
| Palmitoyl peptide² | 0.00055 | 0.00055 | 0.0003 | 0.0003 | 0.00055 | 0.00055 |
| Hydrolyzed soy | — | 4 | — | — | — | — |
| protein | | | | | | |
| N-acetyl glucosamine | 2 | — | 2 | — | 5 | — |
| 1,2-Pentanediol | 1 | 2 | 2 | 3 | 1 | — |
| Triethanolamine | — | — | 0.7 | — | — | — |

Silicone/Oil Phase:

| | | | | | | |
|---|---|---|---|---|---|---|
| Cyclomethicone D5 | 10 | 5 | 5 | 10 | 7.5 | 10 |
| Dow Corning 9040 | — | 10 | 5 | 5 | 7.5 | 5 |
| Silicone elastomer³ | | | | | | |
| KSG-15AP silicone | 5 | — | 5 | 5 | 7.5 | 5 |
| Elastomer⁴ | | | | | | |
| Dimethione/ | — | 2 | 2 | 1 | 2 | 1 |
| dimethiconol | | | | | | |
| Brij 30 | 0.3 | 0.3 | 0.5 | 0.4 | 0.3 | 0.3 |
| Dimethicone 50 csk | 1 | — | — | — | — | — |
| Tetrhydrocurcumin | — | 0.1 | — | — | — | — |
| Salicylic Acid | — | — | — | 0.5 | — | — |
| Phytosterol | — | — | — | — | — | 0.1 |
| PPG-15 Stearyl Ether | 4 | 1 | 2 | 3 | 2 | 2 |
| Dehydroacetic acid | — | — | 0.5 | — | — | — |
| Undecylenoyl | — | — | 0.2 | — | — | — |
| Phenylalanine | | | | | | |
| Ginkgo Biloba Leaf | — | — | 0.1 | 1 | 2 | — |
| Extract | | | | | | |
| Date palm kernal | 4 | 4 | 2 | — | — | 4 |
| extract | | | | | | |
| Vitamin E Acetate | — | 0.5 | 0.1 | 0.1 | — | 0.1 |

Thickener:

| | | | | | | |
|---|---|---|---|---|---|---|
| Polyacrylamide/C13-14 | 2.5 | 2.5 | — | — | — | 3 |
| isoparaffin/laureth-7 | | | | | | |
| Polyquaternium-37 | — | — | — | 1 | — | — |
| Acrylates/C10-30 | — | — | 0.6 | — | 0.5 | — |
| alkyl acrylates | | | | | | |
| crosspolymer | | | | | | |

Undecylenoyl Phenylalanine Premix

| | | | | | | |
|---|---|---|---|---|---|---|
| Undecylenoyl | — | — | — | — | 1 | — |
| Phenylalanine | | | | | | |
| Water | — | — | — | — | 24 | — |
| Triethanolamine | — | — | — | — | 0.5 | — |

Dipalmitoyl Hydroxy-Proline Premix:

| | | | | | | |
|---|---|---|---|---|---|---|
| Water | — | — | — | — | — | 4.4 |
| Triethanolamine | — | — | — | — | — | 0.1 |
| Dipalmitoylhyroxyproline | — | — | — | — | — | 1.0 |

Additional Ingredients:

| | | | | | | |
|---|---|---|---|---|---|---|
| Triethanolamine | — | — | — | — | 0.6 | — |
| Polymethylsil- | 0.5 | 0.5 | 1.0 | 1 | 1 | 0.5 |
| sequioxane | | | | | | |
| Polyethylene | — | 0.5 | 0.5 | 1.0 | — | — |
| Flamenco Summit | — | — | 1.0 | — | — | — |
| Green G30D⁵ | | | | | | |
| Silica | — | — | 1 | 0.5 | — | — |
| Prestige Silk Red⁶ | — | — | — | 1.0 | 1.0 | 1.0 |

¹GLW75CAP-MP, 75% aqueous titanium dioxide dispersion from Kobo
²Available from Sederma
³A silicone elastomer dispersion from Dow Corning Corp
⁴A silicone elastomer dispersion from Shin Etsu,
⁵Titanium dioxide and tin oxide coated mica green interference pigment from Engelhard
⁶Titanium dioxide coated mica red interference pigment from Eckart

In a suitable vessel, combine the water phase ingredients and mix until uniform. In a separate suitable container, combine the silicone/oil phase ingredients and mix until uniform. Separately, prepare the dipalmitoyl hydroxyproline premix and/or undecylenoyl phenylalanine premix by combining the premix ingredients in a suitable container, heat to about 70° C. while stirring, and cool to room temperature while stirring. Add half the thickener and then the silicone/oil phase to the water phase and mill the resulting emulsion (e.g., with a Tekmar T-25). Add the remainder of the thickener, the dipalmitoyl hydroxyproline premix and/or undecylenoyl phenylalanine premix, and then the remaining ingredients to the emulsion while stirring. Once the composition is uniform, pour the product into suitable containers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All publications cited herein are hereby incorporated by reference in their entirety; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising an active component, wherein said composition comprises:
(a) an active component, wherein said active component is selected from the group consisting of In one embodiment, the active component can be selected from the group consisting of:
Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root Extract
Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof;
(b) a dermatologically, orally, or subcutaneous acceptable carrier; and
(c) at least one optional ingredient, wherein said optional ingredient is selected from the group consisting of sugar amine, a vitamin B3 compound, retinoids, peptides, phytosterol, dialkanoyl hydroxyproline, hexamidine, salicylic acid, n-acyl amino acid compounds, sunscreen actives, water soluble vitamins, oil soluble vitamins, their derivatives, their precursors, and combinations thereof.

2. The personal care composition of claim 1, wherein said composition comprises an ingredient selected from the group consisting of niacinamide, hexamidine, and mixtures thereof.

3. A method of marketing a personal care composition, wherein said method comprises:
(a) making available for sale to a potential customer a personal care composition comprising an active component;
(b) communicating to said potential customer that said composition can be helpful in regulating a keratinous tissue condition.

4. A method of marketing a personal care composition, wherein said method comprises:
(a) making available for sale to a potential customer a first personal care composition;
(b) communicating to said potential customer a comparison of said first personal care composition to a second personal care composition, where said second personal care composition comprises an active component.

5. A method of marketing a personal care composition, wherein said method comprises:
(a) providing to a potential customer an article of commerce, wherein said article of commerce comprises:
(1) a container;
(2) a personal care composition contained in said container;
(3) graphics comprising at least two colors on said container;
(b) communicating to said potential customer that said article of commerce is similar to a second article of commerce, wherein said second article of commerce comprises:
(1) a second container;
(2) a second personal care composition contained in said second container, wherein said second personal care composition comprises an active component, wherein said active component is selected from the group consisting of Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root Extract
Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof;
(3) a second set of graphics disposed upon said second container, wherein said a second set of graphics comprises at least one of the same colors as those colors included in said first set of graphics.

6. A method of marketing a personal care composition, wherein said method comprises:
(a) providing to a potential customer an article of commerce, wherein said article of commerce comprises:
(1) a container;
(2) a personal care composition contained in said container;
(3) graphics comprising at least two colors on said container;
(b) locating said article of commerce within close proximity to a second article of commerce, wherein said second article of commerce comprises:
(1) a second container;
(2) a second personal care composition contained in said container, wherein said second personal care composition comprises an active component selected from the group consisting of Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root
Extract
Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof;
(3) a second set of graphics disposed upon said second container, wherein said second set of graphics comprises at least two of the same colors as those colors included in said first set of graphics.

7. A method of marketing a personal care composition, wherein said method comprises:
(a) providing to a potential customer a personal care composition comprising an active component;
(b) communicating to said potential customer that said composition can be helpful in regulating keratinous tissue condition, wherein said keratinous tissue condition is selected from the group consisting of hair growth promotion, hair thickness, hair density, the natural process of skin aging and damage, nail growth, and nail density.

8. A method for regulating keratinous tissue condition, wherein said method comprises topically applying a personal care composition comprising an effective amount of active component to the keratinous tissue of a person seeking regulation thereof.

9. The method of claim 8, wherein said kerationous tissue condition is selected from the group consisting of hair growth promotion, hair thickness, hair density, the natural process of skin aging and damage, nail growth, and nail density.

10. The method of claim 8, wherein said composition comprises an ingredient selected from the group consisting of niacinamide, hexamidine, and mixtures thereof.

11. The method of claim 9, wherein said method comprises applying topically said composition at night.

12. The method of claim 9, wherein said method comprises applying said composition according to a regimen, wherein said regimen comprises:
(a) cleansing keratinous tissue to form cleansed keratinous tissue; and
(b) topically applying said composition to said cleansed keratinous tissue.

13. The regimen of claim 12, wherein said regimen is performed at night.

14. The regimen of claim 12, wherein said regimen is performed before going to bed.

15. The regimen of claim 12, wherein said regimen is performed daily.

16. An article of commerce, wherein said article of commerce comprises:
(1) a container;
(2) a personal care composition contained in said container, wherein said personal care composition comprises an active component, wherein said active component is selected from the group consisting of Sesamol, 5-[(3-aminopropyl)phosphinooxy]-2-(hydroxymethyl)-DHA, (R*,R*)-α,4-dimethyl-α-(4-methyl-3-pentenyl)cyclohex-3-ene-1-methanol, Tannins, [3aR-(3aα,5aβ,9aα,9bβ)]decahydro-3a,6,6,9a-tetramethylnaphth[2,1-b]furan-2(1H)-one, 4-aminobenzoic acid, 6-chlorothymol, (Z)-dihydro-5-(2-octenyl)furan-2(3H)-one, Allyl phenoxyacetate, Cyclohexyl anthranilate, Quaternary ammonium compounds, benzyl-C12-16-alkyldimethyl chlorides, Methylbenzethonium chloride, Docusate sodium, Tributyl citrate, Octahydro-3,8,8-trimethyl-6-methylene-1H-3a,7-methanoazulen-5-ol, 1R-[1α(R*),2β,4aβ,8aα]]-2-hydroxy-α,2,5,5,8a-pentamethyl-α-vinyldecahydronaphthalene-1-propan-1-ol, (2E,6Z)-nona-2,6-dien-1-ol 2-ethyl-4-(2,2,3-trimethyl-3-cyclopenten-1-yl)-2-buten-1-ol, 1-methyl-1-((3S,8S)-1,2,3,4,5,6,7,8-octahydro-3,8-dimethylazulen-5-yl)ethyl acetate, 4-azoniaoctamethylenediammonium trichloride 4-hydroxy-6-methylpyran-4-one, Methyl 2-oxo-2H-pyran-5-carboxylate, Methyl 2-oxo-2H-pyran-3-carboxylate, Tioxolone, Tetrahexyldecyl Ascorbate, Creatine, Menthyl Anthranilate, Ascorbyl Stearate, Vitis Vinifera (Grape) Leaf Extract, Tropaeolum Majus Extract, Hamamelis Virginiana (Witch Hazel) Leaf Extract, Ginkgo Biloba Leaf Extract, Coleus Forskohlii Root Extract, Oleanolic Acid, Xymenynic Acid, Terminalia Arjuna Bark Extract, Trifolium Pratense (Clover) Flower Extract, Coriandrum Sativum (Coriander) Seed Oil, Kaempferia Galanga Root Extract
Arginine/Lysine Polypeptide, Polylysine, Gallic Acid, Scutellaria Baicalensis Root Extract Citrus Reticulata (Tangerine) Peel Extract, Ergothioneine, Emblica Officinalis Fruit, Allantoin Crataegus Monogina Fruit Extract, Schizandra Chinensis Fruit Extract, Capsicum Annuum Extract, Propylene Glycol and Arbutus Unedo Leaf Extract, Rosa Canina Fruit Extract Fucus Vesiculosus Extract, Aesculus Hippocastanum (Horse Chestnut) Seed Extract, Ursolic Acid, Tetrahydropiperine, Centella asiatica, Glycyrrhiza Glabra (Licorice), Curcuma Longa Root Oil, Saccharum Officinarum (Sugar Cane) Extract, Rosmarinic Acid, Zea Mays (Corn) Kernel Extract, Glutamylamidoethyl Indole, Methylsilanol Mannuronate, Phytosterols, Vitis Vinifera (Grape) Seed Oil, Tannic Acid, Ellagic Acid, Triticum Vulgare (Wheat) Germ Extract/Triticum Vulgare (Wheat) Flour Lipids, Hydrolyzed Lupine Protein, Tarchonanthus Camphoratus Oil Symphytum Officinale Leaf Extract, Hibiscus Sabdariffa Flower Extract, Centella Asiatica Extract, Phaseolus Vulgaris (Kidney Bean) Extract, Terminalia Arjuna Bark Extract, Melia Azadirachta Seed Oil, Aesculus Hippocastanum (Horse Chestnut) Seed Extract, Sapindus Trifoliatus Fruit Extract, Thioctic Acid, Rosmarinus Officinalis (Rosemary) Leaf Extract Soy Isoflavones, and derivatives and combinations thereof;
(3) instructions for use of said personal care composition, wherein said instructions direct the user to topically apply said personal care composition to keratinous tissue after cleansing said keratinous tissue.

17. The article of claim 16, wherein said keratinous tissue comprises facial tissue.

18. The article of claim 17, wherein said personal care composition is the composition of claim 1.

19. The article of claim 17, wherein said personal care composition is the composition of claim 2.
